(19) United States
(12) Patent Application Publication
(10) Pub. No.: US 2019/0315695 A1
(43) Pub. Date: Oct. 17, 2019

(54) COMPOUNDS CARRYING NITROGEN-CONTAINING BINDING GROUPS

(71) Applicant: Arkema France, Colombes (FR)

(72) Inventors: Jean-Luc COUTURIER, Lyon (FR); Jean-Francois DEVAUX, Soucieu en Jarrest (FR); Manuel HIDALGO, Brignais (FR)

(73) Assignee: Arkema France, Colombes (FR)

(21) Appl. No.: 16/340,708

(22) PCT Filed: Oct. 11, 2017
(86) PCT No.: PCT/FR2017/052802

§ 371 Date: Apr. 10, 2019

(30) Foreign Application Priority Data
Oct. 12, 2016 (FR) 1659877

(51) Int. Cl. C07D 233/36; C07D 233/32; C08L 7/00; C08L 9/00; C08L 47/00; C08F 253/00; C08F 279/00; C08F 279/02

(52) U.S. Cl. CPC C07D 233/36; C07D 233/32; C08L 7/00; C08F 279/02; C08L 47/00; C08F 253/00; C08F 279/00; C08L 9/00

(57) ABSTRACT

The invention relates to a compound of formula (I), wherein: A1 and A₂ represent, independently of one another, a binding group comprising at least one nitrogen atom; Q₁ and Q₂ represent, independently of one another, a linkage group; and x is an integer between 2 and 6, preferably between 3 and 6. The invention also relates to a rubber composition comprising said compound.

### FIELD OF THE INVENTION

The present invention relates to novel compounds that can be used as modifying agents in rubber compositions, to processes for preparing same, and also to novel rubber compositions comprising these compounds.

### TECHNICAL BACKGROUND

In the industrial field of items produced from rubber compositions, mixtures of polymers with fillers are often used. In order for such mixtures to have good properties, means for improving the dispersion of the fillers within the polymers are constantly being sought. One of the means for achieving this result is the use of coupling agents capable of establishing interactions between the polymer and the filler.

For example, documents FR 2149339 and FR 2206330 describe sulfur-containing compounds comprising two organosilicon end groups, used as coupling agent.

Document WO 2012/007684 describes coupling agents comprising a nitrogenous associative group and a nitrogenous dipole.

Document WO 2012/007685 describes molecules comprising a nitrogenous associative group and an azodicarbonyl group, intended to modify a polymer.

However, these compounds have drawbacks: they are obtained by multi-step synthesis, typically in five steps, and are very expensive to produce. Furthermore, some raw materials required to prepare them, such as mesitol or dichloromethyl methyl ether, are not readily commercially available on a large scale.

Sulfur-containing compounds have also been described in the prior art.

Document WO 03/002653 describes elastomeric compositions comprising a diene elastomer, an inorganic filler and a coupling agent, the latter being a polysilylated organosilicon compound comprising a sulfur-containing group with polythiosulfenamide function.

Document WO 2004/068238 describes silver halide emulsions in which the particles of silver halide have been sensitized in the presence of a polysulfide compound.

The document “Chemical modelling of the thymidylate synthase reaction: evidence for the formation of an exocyclic methylene intermediate from analogues of the covalent ternary complex formed by intramolecular thiol addition to C(6) of 5-aminomethyluracil derivatives”, Paul F. C. van der Melj et al., Tetrahedron Letters, 1988, vol. 29, no. 42, pp 5445-5448, discloses the synthesis of sulfur-containing compounds used as models of the ternary complex in the thymidylate synthase reaction.

Document US 2005/014839 describes disulfide compounds that inhibit histone deacetylases.

Document US 2014/155440 describes bioisosteres of cysteine and cystine for the treatment of schizophrenia and drug addiction.

The document “Precise Discrimination between Butyl and Phenyl Groups in Molecular Aggregates”, Tadashi Endo et al., Chemistry Letters, 1994, pp 2311-2314, describes disulfide compounds comprising two acylurea groups and two butyl, pentyl or phenyl end groups.

Document WO 01/90060 describes disulfide compounds for the treatment of allergies or systemic mastocytosis.

The document “NH Stretching Vibrations and Conformation of Bis[2-(3-substituted ureido)phenyl] disulfides”, A. TS. Antonova, Journal of Molecular Structure, 1989, vol. 197, pp 97-104, describes bis[2-(3-substituted ureido)phenyl]disulfide compounds.

The document “Chelate oxorhenium to assemble new integrin antagonists”, Julien Le Gal et al., Journal of Inorganic Biochemistry, 2011, Vol. 105, pp 880-886, describes the synthesis of oxorhenium complexes that are integrin antagonists, from disulfide compounds especially.

The document “Immunomodulatory action of levamisole—1. Structural analysis and immunomodulating activity of levamisole degradation products”, Kimberly A. Hanson et al., Int. J. Immunopharmac., 1991, vol. 13, no. 6, pp 655-668, discloses levimasole degradation products capable of inhibiting the lymphocyte response, these products being 3-(2-mercaptoethyl)-5-phenylimidazolidine-2-one, 6-phenyl-2,3-dihydroimidazo(2,1-b)thiazole and bis[3-(2-oxo-5-phenylimidazolidin-1-yl)ethyl]disulfide.

There is a real need to provide compounds obtained in few steps, with good yields, from inexpensive and readily available raw materials, these compounds ensuring a good interaction between the polymers and fillers, that is to say making it possible to obtain rubber compositions with good mechanical properties and good wear resistance.

### SUMMARY OF THE INVENTION

The invention relates first and foremost to a compound of formula (I)

A₁-Q₁-Sₓ-Q₂-A₂ (I)

wherein:
A₁ and A₂ represent, independently of one another, an associative group comprising at least one nitrogen atom,
Q₁ and Q₂ represent, independently of one another, a bonding group,
x is an integer ranging from 2 to 6, preferably ranging from 3 to 6.

According to one embodiment, A₁ and A₂ are identical.

According to one embodiment, A₁ and A₂ are independently chosen from the groups imidazolidinone, triazolyl, ureyl, bisureyl and ureidopyrimidyl.

According to one embodiment, A₁ and A₂ independently correspond to one of the following formulae (II) to (VI):

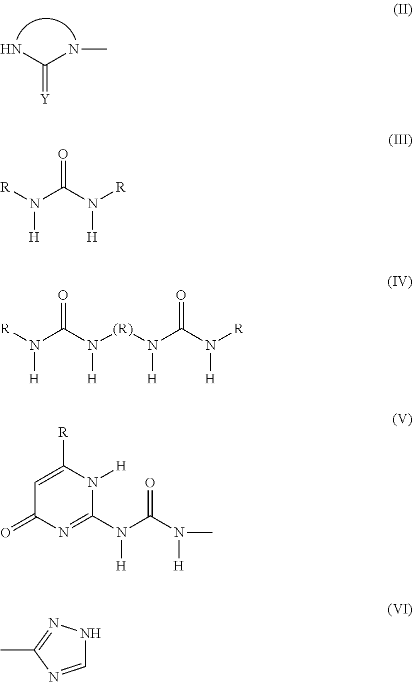

where:
R denotes a hydrocarbon-based group which may optionally contain heteroatoms,
Y denotes an oxygen or sulfur atom, preferably an oxygen atom.

According to one embodiment, at least one of A₁ and A₂, preferably both, is a group of formula (VII):

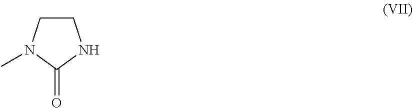

According to one embodiment, Q₁ and Q₂ are independently a linear or branched, substituted or unsubstituted, divalent C1-C24, preferably C1-C10, hydrocarbon-based radical, optionally interrupted and/or substituted with one or more nitrogen or oxygen atoms, and more preferentially an uninterrupted and unsubstituted divalent C1-C6 hydrocarbon-based radical; Q₁ and Q₂ preferably being identical.

According to one embodiment, x is equal to 4.

According to one embodiment, the compound of the invention is chosen from the compounds of following formulae (VIII) to (XI):

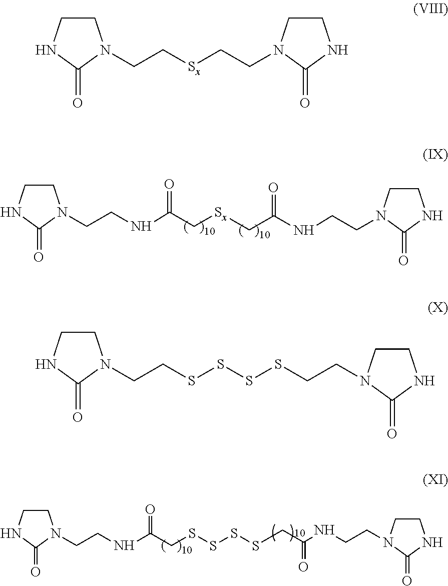

x being an integer ranging from 2 to 4, preferably ranging from 3 to 4, in formulae (VIII) and (IX).

The invention also relates to a mixture of various compounds of formula (I)

A₁-Q₁-Sₓ-Q₂-A₂ (I)

wherein:
A₁ and A₂ represent, independently of one another, an associative group comprising at least one nitrogen atom,
Q₁ and Q₂ represent, independently of one another, a bonding group,
x is an integer ranging from 2 to 6;
   the compounds having different values of x and otherwise being identical, wherein x has a mean value of between 2 and 6.

The invention also relates to a process for preparing a compound as defined above, comprising a step of reacting a sulfur-containing compound with a compound of formula (XII)

A₁-Q₁-Z (XII)

and a compound of formula (XIII)

A₂-Q₂-Z (XIII),

wherein
A₁, A₂, Q₁ and Q₂ have the meanings defined above, and
Z represents a Cl atom or an SH group.

According to one embodiment, the compound of formula (XII) and the compound of formula (XIII) are identical.

According to one embodiment, the sulfur-containing compound is sodium tetrasulfide, Z is a Cl atom and the compound prepared is of formula (I) with x=4; and:
preferably at least one of A₁ and A₂, more preferentially both, is a group of formula (VII):

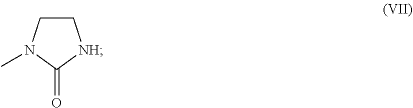

preferably Q₁ and Q₂ are independently a linear or branched divalent C1-C10 hydrocarbon-based radical, more preferentially a linear divalent C2 hydrocarbon-based radical; and/or
preferably the compound of formula A₁-Q₁-Cl and the compound of formula A₂-Q₂-Cl are identical.

According to one embodiment, the sulfur-containing compound is sulfur monochloride, Z is an SH group and the compound prepared is of formula (I) with x=4; and:
preferably at least one of A₁ and A₂, more preferentially both, is a group of formula (VII):

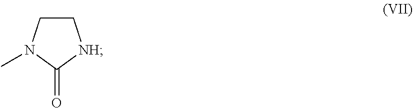

and/or
preferably Q₁ and Q₂ are independently a linear or branched divalent C1-C10 hydrocarbon-based radical, more preferentially a divalent C2 hydrocarbon-based radical; and/or
preferably the compound of formula A₁-Q₁-SH is obtained by reacting a compound of formula A₁-Q₁-Cl with sodium hydrosulfide NaSH; and/or
preferably the compound of formula A₂-Q₂-SH is obtained by reacting a compound of formula A₂-Q₂-Cl with sodium hydrosulfide NaSH; and/or
preferably the compound of formula A₁-Q₁-SH and the compound of formula A₂-Q₂-SH are identical.

According to one embodiment, the sulfur-containing compound is sulfur, Z is an SH group and the compound prepared is of formula (I) with x ranging from 2 to 4; and:
preferably at least one of A₁ and A₂, more preferentially both, is a group of formula (VII):

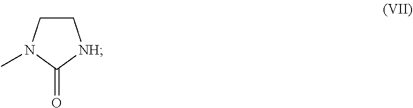

preferably Q₁ and Q₂ are independently a linear or branched divalent C1-C10 hydrocarbon-based radical, more preferentially a linear divalent C2 hydrocarbon-based radical; and/or
preferably the reaction is catalytic; and/or
preferably the compound of formula A₁-Q₁-SH is obtained by reacting a compound of formula A₁-Q₁-Cl with sodium hydrosulfide NaSH; and/or
preferably the compound of formula A₂-Q₂-SH is obtained by reacting a compound of formula A₂-Q₂-Cl with sodium hydrosulfide NaSH; and/or
preferably the compound of formula A₁-Q₁-SH and the compound of formula A₂-Q₂-SH are identical.

The invention also relates to a rubber composition comprising at least one diene elastomer, a reinforcing filler, a chemical crosslinking agent and a modifying agent, optionally already grafted onto the elastomer, said modifying agent being a compound as defined above or a mixture as defined above.

According to one embodiment, the diene elastomer comprises an essentially unsaturated diene elastomer chosen from natural rubber, synthetic polyisoprenes, polybutadienes, butadiene copolymers, isoprene copolymers and mixtures thereof; and/or comprises an essentially saturated elastomer chosen from butyl rubbers, diene/alpha-olefin copolymers such as EPDM, and mixtures thereof.

According to one embodiment, the chemical crosslinking agent comprises from 0.5 to 12 phr of sulfur, preferably from 1 to 10 phr of sulfur, or from 0.01 to 10 phr of one or more peroxide compounds.

According to one embodiment, the content of modifying agent ranges from 0.01 to 50 mol %, preferably from 0.01 mol % to 5 mol %.

The invention also relates to a process for preparing a rubber composition as defined above, comprising one or more steps of thermomechanical kneading of the diene elastomer, the reinforcing filler, the chemical crosslinking agent and the modifying agent, and a step of extruding and calendering.

The invention also relates to an item produced entirely or partly with a rubber composition as defined above, preferably chosen from leaktight seals, thermal or acoustic insulators, cables, sheaths, footwear soles, packagings, coatings (paints, films, cosmetic products), patches (cosmetic or dermopharmaceutical), other systems for trapping and releasing active agents, dressings, elastic clamp collars, vacuum pipes, and pipes and flexible tubing for the transportation of fluids.

The invention also relates to a modified polymer obtained by grafting of a compound as defined above or a mixture thereof as defined above.

According to one embodiment, the polymer is a diene elastomer.

According to one embodiment, the polymer is an essentially unsaturated diene elastomer chosen from natural rubber, synthetic polyisoprenes, polybutadienes, butadiene copolymers, isoprene copolymers and mixtures of these elastomers; or an essentially saturated elastomer chosen from butyl rubbers and diene/alpha-olefin copolymers such as EPDM.

The invention also relates to a process for preparing a modified polymer, comprising a step of grafting a compound as defined above or a mixture as defined above onto a polymer comprising at least one unsaturation.

The present invention makes it possible to overcome the disadvantages of the prior art. It more particularly provides compounds of formula (I) which make it possible to obtain rubber compositions which both have improved properties and a reduced production cost.

The compounds of formula (I) can be produced in few steps, for example from two to four steps, some of which can be carried out in one and the same reactor, and starting from inexpensive raw materials.

Advantageously, the invention makes it possible to obtain rubber compositions which have effective mechanical properties and good wear resistance.

### DESCRIPTION OF EMBODIMENTS OF THE INVENTION

The invention is now described in greater detail and in a nonlimiting manner in the description which follows.

### Compounds of Formula (I)

The invention relates to a compound of formula (I):

A₁-Q₁-Sₓ-Q₂-A₂ (I)

wherein S is a sulfur atom, x is an integer, A₁ and A₂ represent, independently of one another, an associative group comprising at least one nitrogen atom, and Q₁ and Q₂ are bonding groups.

The term “associative groups” is intended to mean groups capable of associating with one another via hydrogen, ionic and/or hydrophobic bonds. According to one preferred embodiment of the invention, they are groups capable of associating via hydrogen bonds.

When the associative groups are capable of associating via hydrogen bonds, each associative group preferably comprises at least one donor “site” and one acceptor site with respect to the hydrogen bond, such that two identical associative groups are self-complementary and can associate with one another by forming at least two hydrogen bonds.

The associative groups according to the invention are also capable of associating, via hydrogen, ionic and/or hydrophobic bonds, with functions present on fillers.

The groups A₁ and A₂ may be different or identical, preferably A₁ and A₂ are identical.

According to a particular embodiment of the invention, the associative groups A₁ and A₂ are independently chosen from imidazolidinone, ureyl, bisureyl, ureidopyrimidyl and triazolyl groups

Preferably, the associative groups A₁ and A₂ independently correspond to one of the following formulae (II) to (VI):

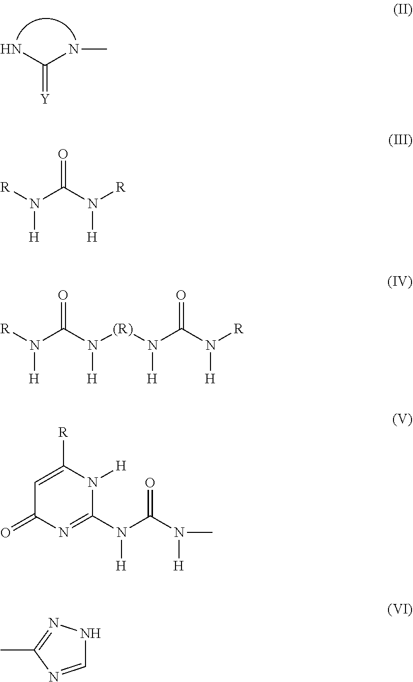

where:
R denotes a linear, branched or cyclic (preferably linear) (preferably C1-C10, even more preferentially C1-C6) hydrocarbon-based group which may optionally contain heteroatoms (and preferably contains no heteroatoms),
Y denotes an oxygen or sulfur atom, preferably an oxygen atom.

In formula (II), the two nitrogen atoms are linked by a divalent organic group, for instance a hydrocarbylene group, such as an alkylene, a substituted alkylene, a cycloalkylene, a substituted cycloalkylene, an arylene or a substituted arylene. The hydrocarbylene group contains from 1 to 10 carbon atoms. The hydrocarbylene group may also contain heteroatoms such as nitrogen, oxygen or sulfur. These heteroatoms may be included in the hydrocarbylene chain or may replace a carbon. Particularly preferably, the group of formula (II) comprises 5 or 6 atoms.

Preferably, the groups A₁ and A₂ are independently a di- or trinitrogenous heterocycle comprising 5 or 6 atoms, preferably dinitrogenous, and comprising at least one carbonyl function.

Even more preferably, the groups A₁ and A₂ are an imidazolidinone group of formula (VII):

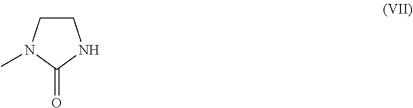

According to a more particular embodiment, A₁ and A₂ are both a group of formula (VII):

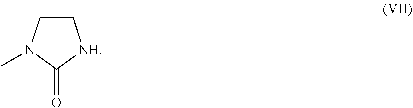

The bonding groups Q₁ and Q₂ may be any divalent radical. They are preferably chosen so as to interfere little or not at all with the associative groups A₁ and A₂.

Said groups Q₁ and Q₂ are then considered to be groups inert with respect to the associative groups A₁ and A₂. “Group inert with respect to the associative groups A₁ and A₂” is intended to mean a group which does not comprise associative functions as defined according to the invention.

The groups Q₁ and Q₂ are preferably independently a linear, branched or cyclic, divalent hydrocarbon-based radical. They may independently contain one or more aromatic radicals, and/or one or more heteroatoms. The divalent hydrocarbon-based radical may optionally be substituted, the substituents preferably being inert with respect to the associative groups A₁ and A₂.

According to one preferred embodiment, the groups Q₁ and Q₂ are independently a linear or branched, substituted or unsubstituted, divalent C1-C24, preferably C1-C10, hydrocarbon-based radical, optionally interrupted and/or substituted with one or more nitrogen or oxygen atoms, and more preferentially an uninterrupted and unsubstituted divalent C1-C6, and more particularly preferably linear, hydrocarbon-based radical.

Q₁ and Q₂ may be different or identical, but preferably Q₁ and Q₂ are identical.

In formula (I) above, x is an integer ranging from 2 to 6.

According to particular embodiments, x is an integer ranging from 2 to 5, or x is an integer ranging from 2 to 4, or x is an integer ranging from 3 to 5, or x is an integer equal to 2 or 3, or x is an integer equal to 3 or 4.

According to other particular embodiments, x is equal to 2 or 3 or 4 or 5 or 6.

According to a particular embodiment, the compound of the invention is chosen from the compounds of following formula (VIII) or (IX):

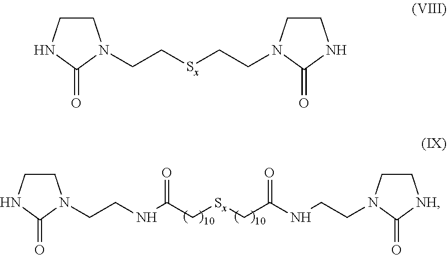

x being an integer ranging from 2 to 6 in formulae (VIII) and (IX), preferably x being an integer ranging from 2 to 5, even more preferentially x being an integer ranging from 2 to 4 and even more preferably, x is an integer equal to 3 or 4.

According to a more particular embodiment, the compound of the invention is chosen from the compounds of following formula (X) or (XI):

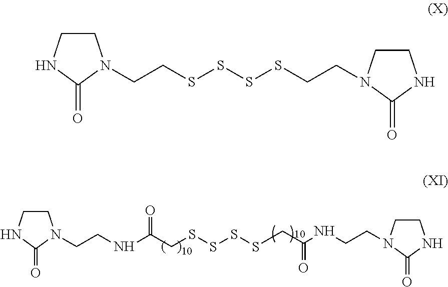

The invention also relates to mixtures of different compounds of formula (I) (and for example of formula (VIII)) with different values of x (the compounds being otherwise identical). For example, the invention relates to mixtures of compounds of formula (I) with x ranging from 2 to 6, or from 2 to 5, or from 2 to 4, the compounds being otherwise identical. The invention also more particularly relates to mixtures of compounds of formula (VIII) with x ranging from 2 to 6, or from 2 to 5, or from 2 to 4, the compounds being otherwise identical. Such a mixture can be considered to be a compound of formula (I) (or respectively of formula (VIII)) with x having a certain statistical distribution and in particular a mean value which is not necessarily a whole number, and which is between 2 and 6 (preferably between 2 and 5, more particularly preferably between 2 and 4).

In particular, certain preparation processes described below result in the production of such compound mixtures.

### Processes for Preparing the Compounds of Formula (I)

The compounds according to the invention may be prepared according to a process comprising, in general, a step of reacting a sulfur-containing compound with a compound of formula (XII)

A₁-Q₁-Z (XII)

and a compound of formula (XIII)

A₂-Q₂-Z (XIII),

wherein
A₁, A₂, Q₁ and Q₂ have the meanings defined above, and
Z represents a Cl atom or an SH group.

The compound of formula (XII) and the compound of formula (XIII) may be different or identical; they are preferably identical. In this case, the process provides the reaction of a certain amount of sulfur-containing compound with a certain amount of the unique compound of formula (XII).

According to one embodiment of the invention, the mixtures of compounds of formula (I) according to the invention, with x on average ranging from 2 to 6, preferably from 2 to 5 and more preferably from 2 to 4, are prepared by a process comprising a step of reacting a sodium polysulfide Na₂Sₓ having a mean x value with a compound of formula A₁-Q₁-Cl and a compound of formula A₂-Q₂-Cl, wherein A₁, A₂, Q₁ and Q₂ have the meanings defined above.

The compound of formula A₁-Q₁-Cl and the compound of formula A₂-Q₂-Cl may be different or identical. They are preferably identical. In this case, the process provides the reaction of a certain amount of sodium polysulfide with a certain amount of the unique compound of formula A₁-Q₁-Cl.

The sodium polysulfide having a mean x value may be prepared by reaction in a solvent between sodium sulfide and sulfur, adapting the respective molar proportions thereof according to the following equation:

Na₂S+(x−1)S→Na₂Sₓ

The reaction for preparing the sodium polysulfide Na₂Sₓ having a mean x value and the reaction thereof with a compound of formula A₁-Q₁-Cl and a compound of formula A₂-Q₂-Cl are preferably carried out in one or more solvents. A broad choice of solvents is possible among the solvents known by those skilled in the art to promote nucleophilic substitutions. For example, use may be made of the following solvents, alone or in a mixture: an alcohol such as methanol, ethanol, 1-propanol, 2-propanol, butanol, an aromatic such as toluene, xylene, an ether such as isopropyl ether, methyl tert-butyl ether, dioxane and tetrahydrofuran.

The reaction of the sodium polysulfide with a compound of formula A₁-Q₁-Cl and a compound of formula A₂-Q₂-Cl may be carried out by adding the compounds of formulae A₁-Q₁-Cl and A₂-Q₂-Cl to a solution of sodium polysulfide or else by adding a solution of sodium polysulfide to a solution of the compounds of formulae A₁-Q₁-Cl and A₂-Q₂-Cl. Alternatively, the solution of sodium polysulfide and the solution of the compounds of formulae A₁-Q₁-Cl and A₂-Q₂-Cl may be added simultaneously to a semi-continuous or continuous reactor.

The temperature of the reaction step may be between room temperature, for example 20° C., and 150° C. and preferably between room temperature, for example 20° C., and 100° C. This step is preferably carried out at the reflux temperature of the solvent at atmospheric pressure.

The molar ratio between the sodium polysulfide Na₂Sₓ having a mean x value and the compounds of formula A₁-Q₁-Cl and A₂-Q₂-Cl is from 0.95 to 1.5, preferably from 1 to 1.2 and more preferentially from 1 to 1.1.

According to a particular embodiment, the reactions are carried out in an anhydrous environment, with anhydrous sodium polysulfide and anhydrous solvents.

The salt formed during the reaction (NaCl) can be removed by filtration and the final product can be isolated by evaporating off the solvent. According to a particular embodiment, a step of washing with water may be carried out in order to remove the inorganic residues from the product.

According to a particular embodiment, the compounds according to the invention of formula (I) with x=4 are prepared by a process comprising a step of reacting sodium tetrasulfide with a compound of formula A₁-Q₁-Cl and a compound of formula A₂-Q₂-Cl, wherein A₁, A₂, Q₁ and Q₂ have the meanings defined above.

Preferably, A₁ and A₂ are identical.

Preferably, at least one of A₁ and A₂ is a group of formula (VII):

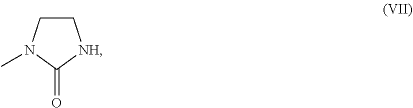

and even more preferentially both A₁ and A₂ are a group of formula (VII).

Preferably, Q₁ and Q₂ are independently a linear or branched, divalent C1-C10 hydrocarbon-based radical, more preferentially a linear divalent C2 hydrocarbon-based radical.

The compound of formula A₁-Q₁-Cl and the compound of formula A₂-Q₂-Cl may be different or identical. They are preferably identical. In this case, the process provides the reaction of a certain amount of sodium tetrasulfide with a certain amount of the unique compound of formula A₁-Q₁-Cl.

The sodium tetrasulfide may be prepared for example by reacting sulfur with sodium sulfide anhydride; the latter may be prepared by reacting sodium ethoxide with hydrogen sulfide. The sodium tetrasulfide is preferably prepared in situ by adding sulfur to an ethanolic solution of sodium sulfide. The final nucleophilic substitution is preferably carried out in the solvent used for preparing the sodium tetrasulfide, that is to say ethanol. The temperature of this step may be between room temperature and the reflux temperature of the solvent. This step is preferably carried out at the reflux temperature of the solvent. The salt formed can be removed by filtration and the final product can be isolated by evaporating off the solvent.

This process may especially be applied to the preparation of the compound of formula (X), according to the following synthesis scheme:

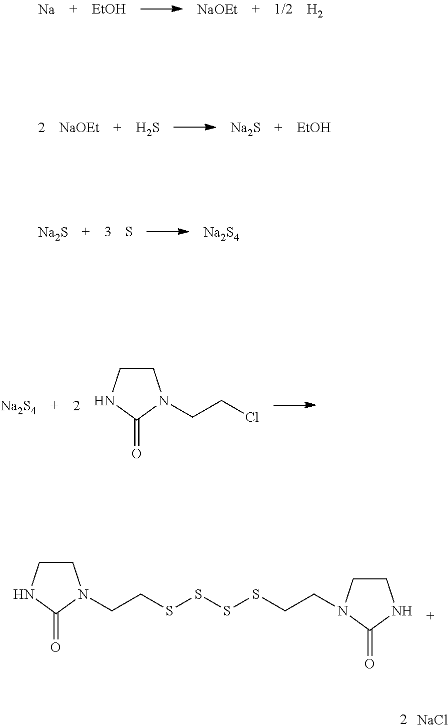

According to another particular embodiment, the compounds according to the invention of formula (I) with x=4 are prepared by a process comprising a step of reacting sulfur monochloride S₂Cl₂ with a compound of formula A₁-Q₁-SH and a compound of formula A₂-Q₂-SH, wherein A₁, A₂, Q₁ and Q₂ have the meanings defined above.

Preferably, A₁ and A₂ are identical.

Preferably, at least one of A₁ and A₂ is a group of formula (VII):

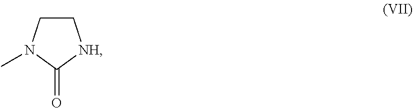

and even more preferentially both A₁ and A₂ are a group of formula (VII).

Preferably, Q₁ and Q₂ are independently a linear or branched, divalent C1-C10 hydrocarbon-based radical, more preferentially a linear divalent C2 hydrocarbon-based radical.

More preferably still, Q₁ and Q₂ are identical.

Preferably, the compound of formula A₁-Q₁-SH is obtained by reacting a compound of formula A₁-Q₁-Cl with sodium hydrosulfide NaSH. It may also be obtained by an esterification or amidation reaction from compounds of A₁-OH or A₁-NH₂ type with a compound of HOOC-Q₁-SH type (cf. example 3 below).

Preferably, the compound of formula A₂-Q₂-SH is obtained by reacting a compound of formula A₂-Q₂-Cl with sodium hydrosulfide NaSH. It may also be obtained by an esterification or amidation reaction from compounds of A₂-OH or A₂-NH₂ type with a compound of HOOC-Q₂-SH type (cf. example 3 below).

The compound of formula A₁-Q₁-SH and the compound of formula A₂-Q₂-SH may be different or identical; preferably, the compound of formula A₁-Q₁-SH and the compound of formula A₂-Q₂-SH are identical. In this case, the process provides the reaction of a certain amount of sulfur monochloride with a certain amount of the unique compound of formula A₁-Q₁-SH.

This process may be carried out in solvent medium, preferably tetrahydrofuran, at a temperature of between −10° C. and 30° C., preferably of approximately 0° C.

The compound of formula (X) may thus be prepared from an imidazolidinone mercaptan and sulfur monochloride, according to the following synthesis scheme:

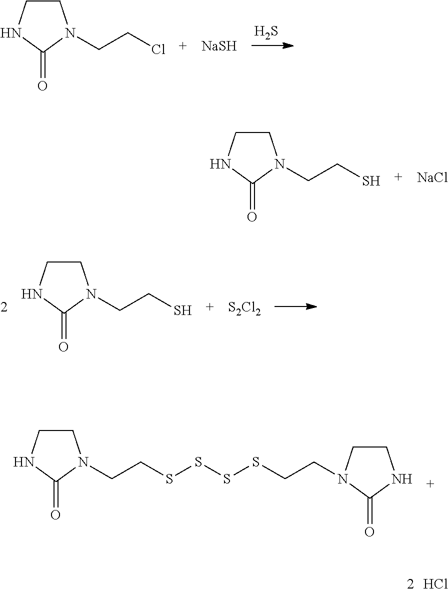

According to another particular embodiment, the compounds according to the invention of formula (I) with x ranging from 2 to 6, preferably from 2 to 5, and more particularly from 2 to 4, are prepared by a process comprising a step of reacting sulfur with a compound of formula A₁-Q₁-SH and a compound of formula A₂-Q₂-SH, wherein A₁, A₂, Q₁ and Q₂ have the meanings defined above.

Preferably, A₁ and A₂ are identical.

Preferably, at least one of A and A₂ is a group of formula (VII):

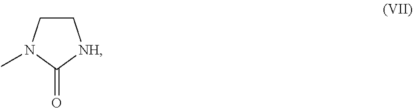

and even more preferentially both A₁ and A₂ are a group of formula (VII).

Preferably, Q₁ and Q₂ are independently a linear or branched, divalent C1-C10 hydrocarbon-based radical, more preferentially a linear divalent C2 hydrocarbon-based radical. Preferably, Q₁ and Q₂ are identical.

Preferably, the compound of formula A₁-Q₁-SH is obtained by reacting a compound of formula A₁-Q₁-Cl with sodium hydrosulfide NaSH. It may also be obtained by an esterification or amidation reaction from compounds of A₁-OH or A₁-NH₂ type with a compound of HOOC-Q₁-SH type (cf. example 3 below).

Preferably, the compound of formula A₂-Q₂-SH is obtained by reacting a compound of formula A₂-Q₂-Cl with sodium hydrosulfide NaSH. It may also be obtained by an esterification or amidation reaction from compounds of A₂-OH or A₂-NH₂ type with a compound of HOOC-Q₂-SH type (cf. example 3 below).

The compound of formula A₁-Q₁-SH and the compound of formula A₂-Q₂-SH may be different or identical; they are preferably identical. In this case, the process provides the reaction of a certain amount of sulfur with a certain amount of the unique compound of formula A₁-Q₁-SH.

Preferably, the reaction is catalytic. The reaction step may be carried out in the presence of a catalyst, which may especially consist of a combination of a mercaptan with an alkene oxide, preferably ethylene oxide, and an alkaline base, preferably sodium hydroxide.

A reaction solvent may be used, especially if the melting point of the polysulfide is greater than 100° C.

The implementation of such a process generally makes it possible to obtain a mixture of polysulfide compounds having a distribution of the number of sulfur atoms ranging from 2 to 6, more particularly from 2 to 5, and principally from 2 to 4.

In particular, the compound of formula (VIII) with x ranging from 2 to 6, more particularly from 2 to 5, and principally from 2 to 4, may be prepared from an imidazolidinone mercaptan and sulfur according to the following synthesis scheme:

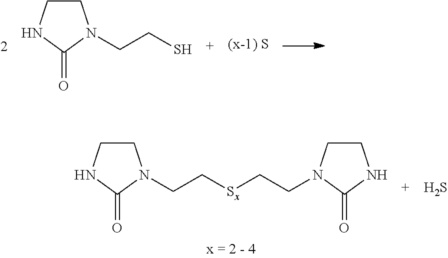

### Applications

The invention also relates to a rubber composition comprising at least one diene elastomer, a reinforcing filler, a chemical crosslinking agent and a modifying agent, optionally already grafted onto the elastomer, said modifying agent being a compound according to the invention as described above.

According to one embodiment, the rubber composition is a simple (not crosslinked or vulcanized) mixture of the constituents above.

According to one embodiment, the rubber composition is a crosslinked or vulcanized mixture based on the constituents above.

In the present description, unless expressly indicated otherwise, all the percentages (%) indicated are by weight.

One of the components of the rubber composition according to the invention is a diene elastomer.

The diene elastomers can be categorized, in a known manner, in two categories, those termed essentially unsaturated and those termed essentially saturated. These two categories of diene elastomers can be envisioned in the context of the invention.

An essentially saturated diene elastomer has a low or very low content of moieties or units of diene origin (conjugated dienes) which is always less than 15% (by mol). Thus, for example, butyl rubbers or diene/alpha-olefin copolymers, such as EPDM (ethylene-propylene-diene monomer) come under the definition of essentially saturated diene elastomers

Conversely, the term “essentially unsaturated diene elastomer” is intended to mean a diene elastomer at least partly derived from conjugated diene elastomers, having a content of moieties or units of diene origin (conjugated dienes) which is greater than 15% (by mol). In the category of essentially unsaturated diene elastomers, the term “highly unsaturated diene elastomer” is in particular intended to mean a diene elastomer having a content of moieties of diene origin (conjugated dienes) which is greater than 50% (by mol).

The term “diene elastomer which can be used in the invention” is intended to mean more particularly:

(a) any homopolymer obtained by polymerization of a conjugated diene monomer having from 4 to 12 carbon atoms;

(b) any copolymer obtained by copolymerization of one or more conjugated dienes with one another or with one or more vinyl aromatic compounds having from 8 to 20 carbon atoms;

(c) any ternary copolymer obtained by copolymerization of ethylene, of an α-olefin having from 3 to 6 carbon atoms with a nonconjugated diene monomer having from 6 to 12 carbon atoms, for instance the elastomers obtained from ethylene, from propylene with a nonconjugated diene monomer of the abovementioned type, such as especially 1,4-hexadiene, ethylidene norbornene, dicyclopentadiene; such polymers are described in particular in documents WO 2004/035639A1 and US 2005/0239639A1;

(d) any copolymer of isobutene and of isoprene (butyl rubber), and also the halogenated versions, in particular chlorinated or brominated versions, of copolymers of this type.

The diene elastomers of the highly unsaturated type, in particular of type (a) or (b) above, are preferred.

Suitable conjugated dienes are especially 1,3-butadiene, 2-methyl-1,3-butadiene, 2,3-di(C₁-C₅)alkyl-1,3-butadienes, such as for example 2,3-dimethyl-1,3-butadiene, 2,3-diethyl-1,3-butadiene, 2-methyl-3-ethyl-1,3-butadiene, 2-methyl-3-isopropyl-1,3-butadiene, an aryl-1,3-butadiene, 1,3-pentadiene and 2,4-hexadiene.

Suitable vinyl aromatic compounds are for example styrene, ortho-, meta-, para-methylstyrene, the commercial “vinyl-toluene” mixture, para-tert-butylstyrene, methoxystyrenes, chlorostyrenes, vinylmesitylene, divinylbenzene and vinylnaphthalene.

The copolymers may contain between 99% and 20% by weight of diene units and between 1% and 80% by weight of vinyl aromatic units. The elastomers may have any microstructure, which depends on the polymerization conditions used, especially on the presence or absence of a modifying and/or randomizing agent and on the amounts of randomizing modifying agent used. The elastomers may for example be block, random, sequenced or micro-sequenced elastomers, and may be prepared in dispersion, in emulsion or in solution; they may be coupled and/or star-branched or else functionalized with a coupling and/or star-branching or functionalizing agent.

Particularly suitable are the diene elastomers chosen from the group consisting of polybutadienes (BR), synthetic polyisoprenes (IR), natural rubber (NR), butadiene copolymers, isoprene copolymers and mixtures of these elastomers. Such copolymers are more preferentially chosen from the group consisting of butadiene-styrene copolymers (SBR), isoprene-butadiene copolymers (BIR), isoprene-styrene copolymers (SIR), isoprene-butadiene-styrene copolymers (SBIR) and mixtures of such copolymers.

The rubber composition according to the invention also comprises at least the modifying agent which is a compound of formula (I) or one of the preferred variants thereof described above. The diene elastomer may be grafted by the modifying agent prior to its introduction into the rubber composition, or else may be grafted by reaction with the modifying agent during the production of the composition.

The rubber composition according to the invention may thus contain a single diene elastomer grafted by the modifying agent (either grafted prior to its introduction into the composition, or grafted by reaction with the modifying agent during the production of the composition), or a mixture of several diene elastomers which are all grafted, or some of which are grafted and others not.

The other diene elastomer(s) used as a blend with the grafted elastomer according to the invention are conventional diene elastomers as described above, whether star-branched, coupled, functionalized or nonfunctionalized. These elastomers are then present in the matrix at a content of between 0 and 60 phr (the limits of this range being excluded), preferentially at a content ranging from more than 0 to 50 phr, even more preferentially from more than 0 to 30 phr.

In the case of a blend with at least one other diene elastomer, the weight fraction of grafted elastomer according to the invention in the elastomeric matrix is predominant and preferably greater than or equal to 50% by weight of the total weight of the matrix. The term “predominant weight fraction” refers according to the invention to the highest weight fraction of the blend.

It will be noted that, the lower the proportion of said supplementary elastomer(s) in the composition according to the invention, the greater the improvement in the properties of the rubber composition according to the invention.

The grafted diene elastomer(s) according to the invention can be used in combination with any other type of synthetic elastomer other than a diene elastomer, or even with polymers other than elastomers, for example thermoplastic polymers.

According to a preferred embodiment, the content of modifying agent ranges from 0.01 to 50 mol %, preferably from 0.01 mol % to 5 mol %.

In the remainder of the text, the term “content of modifying agent” present in a rubber composition, expressed as molar percentage, is intended to mean the number of molecules of modifying agent present in the composition per hundred moieties of diene elastomer of the composition, whether they are, without distinction, diene or non-diene moieties.

For example, if the content of modifying agent on an SBR is 0.20 mol %, this means that there is 0.20 moiety derived from modifying agent per 100 SBR styrene and butadiene moieties.

In the case where both an elastomer already grafted by the modifying agent and a diene elastomer not grafted by a modifying agent are used in the composition, the content of modifying agent represents the number of molecules of modifying agent grafted per 100 diene elastomer moieties, the number of moeties taking into account the two elastomers (grafted and nongrafted), assuming that other molecules of modifying agent not already grafted have not been added to the composition.

Another component of the rubber composition according to the invention is the reinforcing filler.

Use may be made of any type of reinforcing filler known for its capacities to reinforce a rubber composition, for example a reinforcing organic filler such as carbon black, a reinforcing inorganic filler such as silica, or else a blend of these two types of filler, especially a blend of carbon black and silica. As other reinforcing fillers, use may also be made of cellulose-based fillers, talc, calcium carbonate, mica or wollastonite, glass or metal oxides or hydrates. Preferably, a reinforcing inorganic filler is present.

All the carbon blacks are suitable carbon blacks, especially those of the HAF, ISAF or SAF type. Use may also be made, depending on the intended applications, of the higher series blacks FF, FEF, GPF, SRF. The carbon blacks could for example already be incorporated into the diene elastomer in the form of a masterbatch, before or after grafting and preferably after grafting (see for example documents WO 97/36724 or WO 99/16600).

As examples of organic fillers other than carbon blacks, mention may be made of the functionalized polyvinyl aromatic organic fillers as described in documents WO 2006/069792 and WO 2006/069793.

In the present application, the term “reinforcing inorganic filler” should be understood to mean, by definition, any mineral or inorganic filler, as opposed to carbon black, capable of reinforcing by itself a rubber composition, without any means other than an intermediate coupling agent; such a filler is generally characterized, in a known manner, by the presence of hydroxyl groups at its surface.

The physical state in which the reinforcing inorganic filler is provided is unimportant, whether it is in powder, microbead, granule or bead form or any other suitable densified form. Of course, the term “reinforcing inorganic filler” is also intended to mean mixtures of various reinforcing inorganic fillers, in particular of highly dispersible siliceous and/or aluminous fillers as described hereinafter.

Suitable reinforcing inorganic fillers are especially mineral fillers of the siliceous type, in particular silica (SiO₂), or of the aluminous type, in particular alumina (Al₂O₃). According to the invention, the content of reinforcing filler in the composition is between 30 and 150 phr, more preferentially between 50 and 120 phr. The optimum is different depending on the particular applications intended.

According to one particularly preferred embodiment, a mineral filler of siliceous type is present preferably in a content of from 30 to 150 phr.

According to one embodiment, the reinforcing filler comprises predominantly silica, the content of carbon black present in the composition preferably being between 2 and 20 phr.

According to another embodiment of the invention, the reinforcing filler comprises predominantly carbon black, or even exclusively consists of carbon black.

In order to couple the reinforcing inorganic filler to the diene elastomer, it is possible to optionally include, in the composition, an at least bifunctional coupling agent (or bonding agent) intended to ensure a sufficient connection, of chemical and/or physical nature, between the inorganic filler (surface of its particles) and the diene elastomer, in particular bifunctional organosilanes or polyorganosiloxanes, for example bis(3-triethoxysilylpropyl) tetrasulfide.

Use may especially be made, in a known manner, of the polysulfide-containing silanes, termed symmetrical or asymmetrical depending on their particular structure, as described for example in documents WO 03/002648 and WO 03/002649.

The content of coupling agent, when it is present, is preferentially between 4 and 12 phr, more preferentially between 3 and 8 phr.

Alternatively, the composition may be free of coupling agent, the coupling of the reinforcing inorganic filler to the diene elastomer being provided solely by the modifying agent described above.

As filler equivalent to the reinforcing inorganic filler described in the present paragraph, use may also be made of a reinforcing filler of another nature, especially organic, provided that this reinforcing filler is covered with an inorganic layer such as silica, or else comprises, at its surface, functional sites, especially hydroxyl sites, requiring coupling to establish the bond between the filler and the elastomer.

Another component of the rubber composition according to the invention is the chemical crosslinking agent.

The chemical crosslinking allows the formation of covalent bonds between the elastomer chains. The chemical crosslinking can be carried out especially by means of a vulcanization system or else by means of peroxide compounds.

The vulcanization system per se is based on sulfur (or on a sulfur-donating agent) and on a primary vulcanization accelerator. Various known secondary accelerators or vulcanization activators, such as zinc oxide, stearic acid or equivalent compounds, or guanidine derivatives (in particular diphenylguanidine) may be added to this basic vulcanization system.

The sulfur is used in a preferential content of between 0.5 and 12 phr, in particular between 1 and 10 phr. The primary vulcanization accelerator is used in a preferential content of between 0.5 and 10 phr, more preferentially of between 0.5 and 5.0 phr.

Any compound capable of acting as a vulcanization accelerator for diene elastomers in the presence of sulfur, especially accelerators of thiazole type and also derivatives thereof, and accelerators of thiuram or zinc dithiocarbamate type, may be used as (primary or secondary) accelerator. A primary accelerator of the sulfenamide type is preferably used.

When the chemical crosslinking is carried out by means of one or more peroxide compounds, said peroxide compound(s) represent(s) from 0.01 to 10 phr.

As peroxide compounds that can be used as chemical crosslinking system, mention may be made of acyl peroxides, for example benzoyl peroxide or p-chlorobenzoyl peroxide, ketone peroxides, for example methyl ethyl ketone peroxide, peroxyesters, for example tert-butyl peroxyacetate, tert-butyl peroxybenzoate and tert-butyl peroxyphthalate, alkyl peroxides, for example dicumyl peroxide, di-tert-butyl peroxybenzoate and 1,3-bis(tert-butyl peroxyisopropyl)benzene, hydroperoxides, for example tert-butyl hydroperoxide.

The rubber composition according to the invention may be a single-phase or polyphase system.

The rubber composition according to the invention may also comprise all or some of the usual additives customarily used in rubber compositions, for instance petroleum fractions, solvents, plasticizers or extender oils, whether the latter are of aromatic or nonaromatic nature, pigments and/or dyes, tackifying resins, processing aids, lubricants, anti-radiation (anti-UV) additives, protective agents such as anti-ozone waxes (such as Ozone Wax C32 ST), chemical antiozonants, antioxidants (such as 6-paraphenylenediamine), anti-fatigue agents, reinforcing resins, methylene acceptors (for example phenolic novolac resin) or methylene donors (for example HMT or H3M) as described for example in document WO 02/10269, and also adhesion promoters (cobalt salts for example).

In particular, additives that can be added to the material according to the invention are especially:
lubricants, such as stearic acid and esters thereof, waxy esters, polyethylene waxes, paraffin or acrylic lubricants;
dyes;
mineral or organic pigments, such as those described in the document “Plastics Additives and Modifiers Handbook, Section VIII, Colorants”, J. Edenbaum, published by Van Nostrand, p. 884-954. By way of example of pigments that can be used, mention may be made of carbon black, titanium dioxide, clay, metal particles or treated mica particles of the Iriodin® brand-name sold by Merck;
plasticizers;
heat and/or UV stabilizers, such as tin, lead, zinc, cadmium, barium or sodium stearates, including Thermolite® from Arkema;
co-stabilizers such as epoxidized natural oils;
antioxidants, for example phenolic, sulfur-containing or phosphite antioxidants;
antistatic agents;
fungicides and biocides;
swelling agents used to produce expanded articles, such as azodicarbonamides, azobisisobutyronitrile, diethyl azobisisobutyrate;
fire retardants, including antimony trioxide, zinc borate and brominated or chlorinated phosphate esters;
solvents; and
mixtures thereof.

Preferably, the rubber composition according to the invention comprises, as nonaromatic or very weakly aromatic preferential plasticizing agent, at least one compound chosen from the group consisting of naphthenic oils, paraffinic oils, MES oils, TDAE oils, glycerol esters (in particular trioleates), hydrocarbon-based plasticizing resins having a high glass transition temperature (Tg) of preferably greater than 30° C., and mixtures of such compounds.

The composition according to the invention may also contain, in addition to the coupling agents, reinforcing inorganic filler coupling activators or more generally processing aids capable, in a known manner, by virtue of an improvement in the dispersion of the inorganic filler in the rubber matrix and of a decrease in the viscosity of the compositions, of improving the processing capability thereof in the raw state.

The invention also relates to a process for preparing a rubber composition according to the invention, comprising one or more steps of thermomechanical kneading of the diene elastomer, the reinforcing filler, the chemical crosslinking agent and the modifying agent, and a step of extruding and calendering, or else of extrusion-blow molding, conventional molding, injection-molding, rotational molding or thermoforming.

The rubber composition according to the invention may especially be produced in a suitable mixer using two successive preparation phases: a phase of thermomechanical working or kneading (sometimes termed “non-productive phase”) at high temperature, up to a maximum temperature of between 130° C. and 200° C., preferably between 145° C. and 185° C., followed by a second phase (sometimes termed “productive phase”) at a lower temperature, typically less than 120° C., for example between 60° C. and 100° C.: this is a finishing phase during which the chemical crosslinking system is incorporated.

Generally, all the basic constituents of the composition, with the exception of the chemical crosslinking system, namely the reinforcing filler(s), the coupling agent where appropriate, are incorporated intimately, by kneading, into the diene elastomer(s) during the first, non-productive, phase, that is to say that at least these different bases constituents are introduced into the mixer and thermomechanically kneaded until the maximum temperature of between 130° C. and 200° C., preferably of between 145° C. and 185° C., is reached.

According to a second embodiment of the invention, the diene elastomer is grafted with the modifying agent prior to the production of the rubber composition.

Thus, in this case, it is the grafted diene elastomer which is introduced during the first phase, termed non-productive. Thus, according to this first embodiment of the process, said process comprises the following steps:
modifying the diene elastomer post-polymerization or in solution or in bulk by grafting of a modifying agent as described above;
incorporating, into the diene elastomer thus grafted with the modifying agent, the reinforcing filler and all the basic constituents of the composition, with the exception of the chemical crosslinking system, by thermomechanically kneading the whole mixture, in one or more steps, until a maximum temperature of between 130° C. and 200° C., preferably between 145° C. and 185° C., is reached;
cooling the whole mixture to a temperature of less than 100° C.;
then incorporating the chemical crosslinking agent;
kneading the whole mixture until a maximum temperature of less than 120° C. is reached;
extruding or calendering the rubber composition thus obtained.

According to a second embodiment of the invention, the grafting of the diene elastomer with the modifying agent is carried out concomitantly with the production of the rubber composition. In this case, both the as yet non-grafted diene elastomer and the modifying agent are introduced during the non-productive first phase.

Preferentially, the reinforcing filler can then be subsequently added during this same non-productive phase in order to prevent any unwanted reaction with the modifying agent.

Thus, according to this second embodiment of the process, said process comprises the following steps:
incorporating, into the diene elastomer, a modifying agent as described above, at a temperature and for a period of time such that the grafting yield is preferably greater than 60%, more preferentially greater than 80%, and, preferably subsequently, the reinforcing filler, and also all the basic constituents of the composition, with the exception of the chemical crosslinking system, by thermomechanically kneading the whole mixture, in one or more steps, until a maximum temperature of between 130° C. and 200° C., preferably between 145° C. and 185° C., is reached;
cooling the whole mixture to a temperature of less than 100° C.;
then incorporating the chemical crosslinking agent;
kneading the whole mixture until a maximum temperature of less than 120° C. is reached;
extruding or calendering the rubber composition thus obtained.

The grafting of the modifying agent can be carried out in bulk, for example in an internal mixer or an external mixer such as a cylinder mixer. The grafting is then carried out either at a temperature of the external mixer or of the internal mixer of less than 60° C., followed by a step of grafting reaction in a press or in an oven at temperatures ranging from 80° C. to 200° C., or at a temperature of the external mixer or of the internal mixer of greater than 60° C. without subsequent heat treatment.

The compositions obtained in this way are calendered either in the form of slabs (thickness of 2 to 3 mm) or thin sheets of rubber for the measurement of their physical or mechanical properties, or in the form of profiled elements which can be used directly, after cutting and/or assembling to the desired dimensions, for example as finished or semi-finished products.

The invention makes it possible in particular to obtain leaktight seals, thermal or acoustic insulators, cables, sheaths, footwear soles, packagings, coatings (paints, films, cosmetic products), patches (cosmetic or demopharmaceutical), or other systems for trapping and releasing active agents, dressings, elastic clamp collars, vacuum pipes, and pipes and flexible tubing for the transportation of fluids and, generally speaking, parts that need to have elastic behavior while having good flexibility, good resistance to fatigue, impacts and tearing. These materials may also form part of adhesive or cosmetic compositions or ink, varnish or paint formulations.

### Modified Polymers

Another subject of the invention is a modified polymer obtained by grafting a compound according to the invention of formula (I) or corresponding to one of the preferred embodiments.

Preferably, the polymer contains at least one unsaturation or double bond capable of reacting with the compound according to the invention.

Preferably, the polymers in question are diene elastomers, as defined above.

According to the invention, the polymer having at least one unsaturation or double bond is modified by grafting a compound of formula (I) as defined above, also called modifying agent.

According to a preferred embodiment, the content of modifying agent ranges from 0.01 to 50 mol %, preferably from 0.01 mol % to 5 mol %.

The invention also relates to a process for producing a modified polymer, comprising a step of grafting a compound according to the invention as defined above onto a polymer comprising at least one unsaturation.

The accepted mechanism for the grafting is homolytic cleavage of the polysulfide, followed by radical addition of S° radicals on the double bonds of the polymer.

The grafting of the modifying agent can be carried out in bulk, for example in an internal mixer or an external mixer such as a cylinder mixer, or in solution. The grafting process may be carried out in solution in continuous or batchwise mode. The polymer modified in this way may be separated from its solution by any type of means known to those skilled in the art and in particular by a steam bubbling operation.

For example, the grafting step may be carried out in the melt state, for example in an extruder or an internal mixer, at a temperature which may range from 50° C. to 300° C. and preferably from 200 to 280° C. The modifying agent may be mixed with the polymer alone or using an additive that enables the impregnation of the solid polymer grains by the pre-melted modifying agent. Before introduction into the extruder or the mixer, the solid mixture may be made more homogeneous by refrigeration so as to solidify the modifying agent. It is also possible to meter the latter into the extruder or the mixer after the polymer to be grafted has begun to melt. The time at the grafting temperature can range from 30 seconds to 5 hours. The modifying agent can be introduced into the extruder in the form of a masterbatch in a polymer which, preferably, can be the polymer to be grafted. According to this method of introduction, the masterbatch may comprise up to 30% by weight of the modifying agent; the masterbatch is subsequently diluted in the polymer to be grafted during the grafting operation.

According to another possibility, the grafting can be carried out by solvent-phase reaction, for example in anhydrous chloroform. In this case (anhydrous chloroform), the reaction temperature can range from 5° C. to 75° C., for a period of time ranging from a few minutes to one day and at concentrations of polymer before grafting of between 1% and 50% by weight, relative to the total weight of the solution.

The number of associative groups introduced onto the polymer is adjusted so as to obtain materials which have good dimensional stability and good mechanical properties by virtue of permanent chemical crosslinking, while at the same time being easier to process and having particular properties, such as for example mechanical properties which can be adjusted, owing to the introduction of a different method of cross-linking (non-permanent) capable of evolving as a function of the parameters of the environment in which said materials are used, such as, for example, the characteristic stress time or temperature.

For example, the average number of associative groups per polymer chain can be between 1 and 200.

Thus, the ratio between the percentage of permanent covalent bond crosslinking bridges and the percentage of noncovalent bond crosslinking bridges is advantageously between 99/1 and 1/99, and preferably between 90/10 and 20/80.

### EXAMPLES

The following examples illustrate the invention without limiting it.

### Example 1—Synthesis of the Compound of Formula (X) Using Na₂S₄

1-(2-Chloroethyl)imidazolidin-2-one is prepared according to example 1b of document WO 2012/007684.

15.2 g of sodium (0.66 mol) are introduced into a 500 ml glass reactor fitted with a reflux condenser and flushed with nitrogen. 200 g of ethanol are slowly added, then the mixture is left at the reflux of ethanol for approximately 1 h until the sodium has entirely dissolved.

The mixture is cooled to 40° C., then 7.4 normal liters, or 11.2 g, of H₂S (0.33 mol) are introduced into the reaction mixture via a diffuser over a period of approximately 1 hour.

At the end of the addition of H₂S, the mixture is cooled to 25° C., and 31.7 g of sulfur (0.99 mol) are added. The mixture is allowed to react for 15 minutes, then nitrogen is bubbled into the reaction mixture before heating to the reflux of ethanol. 98.1 g of 1-(2-chloroethyl)imidazolidin-2-one (0.66 mol) are then added over a period of 1 hour, then the mixture is left to react for 2 hours at reflux.

The reaction mixture is cooled to room temperature then filtered. The precipitate is washed with 100 g of ethanol. The filtrates are brought together and evaporated under vacuum. 104 g of the compound of formula (I) are obtained (yield: 89%).

### Example 2—Synthesis of the Compound of Formula (X) Using S₂Cl₂

300 g of methanol and 13.6 g of NaOH (0.34 mol) are charged into a 1 l autoclave. The autoclave is closed and H₂S is introduced with stirring at a flow rate of 12 g/h until a pressure of 20 bar is reached. At 20° C., 50 g of 1-(2-chloroethyl)imidazolidin-2-one (0.34 mol) dissolved in 200 g of methanol are then introduced over a period of one hour. At the end of the addition, the mixture is left to react for 1 hour at 80° C. The autoclave is cooled to room temperature then depressurized. The reaction mixture is degassed with nitrogen then filtered. The filtrate is concentrated by a factor of 5, then the precipitate is removed by filtration. The filtrate is evaporated under vacuum to give 1-(2-mercaptoethyl)imidazolidin-2-one.

The 1-(2-mercaptoethyl)imidazolidin-2-one is dissolved in 400 g of tetrahydrofuran (THF) and transferred into a 1 l glass reactor. 34.4 g of triethylamine (0.34 mol) are added. The mixture is cooled to 0° C., then 19.9 g of S₂Cl₂ (0.17 mol) are slowly added. At the end of the addition, the mixture is allowed to return to room temperature. The reaction mixture is filtered then the THF is evaporated under vacuum. 47 g of the compound of formula (X) are obtained (yield=78%).

### Example 3—Synthesis of the Compound of Formula (IX) Using S

75 g of 1-(2-aminoethyl)imidazolidin-2-one (0.58 mol) and 126.6 g of 11-mercaptoundecanoic acid (0.58 mol) are charged in a 500 ml reactor. The mixture is heated to 160° C. under nitrogen and left to react for 6 hours, with removal of the water formed to a Dean-Stark apparatus. The mixture is cooled to room temperature and the corresponding amide is obtained quantitatively: 11-mercapto-N-[2-(2-oxoimidazolidin-1-yl)ethyl]undecaneamide (melting point=99-103° C.).

150 g of the amide obtained previously (0.46 mol) and 150 g of sodium ethoxide at 0.1% in ethanol are charged in a 500 ml reactor. The mixture is heated to reflux, then 29.5 g of sulfur (0.92 mol) are added over a period of 1 hour. At the end of the addition, the reaction mixture is subjected to bubbling with nitrogen for 1 hour while remaining at the reflux of ethanol. The ethanol is evaporated under vacuum to give 168 g of the compound of formula (IX) which is in the form of a mixture of polysulfides having a mean value of 3 (xₘₑₐₙ=3).

### Example 4—Synthesis of a Mixture of Compounds of Formula (VIII) Using Na₂Sₓ

28.1 g of anhydrous sodium sulfide (0.36 mol), 516 g of toluene and 207 g of anhydrous ethanol are introduced into a 1 l glass reactor fitted with a reflux condenser and flushed with nitrogen. 34.7 g of sulfur (1.08 mol) are added with stirring at room temperature, then the mixture is brought to reflux at atmospheric pressure for 2 hours. A mixture of 101 g of 1-(2-chloroethyl)-imidazolidin-2-one (0.34 mol) in 206 g of anhydrous ethanol is then added and left at reflux for 4 hours.

The reaction mixture is cooled to room temperature then filtered. The precipitate is washed with 100 g of ethanol. The filtrates are brought together and evaporated under vacuum. 118 g of a crude product are obtained, which is taken up in dichloromethane and washed with 100 g of water. After settling, the organic phase is evaporated under vacuum. A solid product is recovered. NMR analysis indicates that a distribution of compounds of formula (VIII) has been obtained, with 22 mol % of compounds of formula (VIII) with a sulfur value equal to 2, 26 mol % of compounds of formula (VIII) with a sulfur value equal to 3, 51 mol % of compounds of formula (VIII) with a sulfur value of greater than or equal to 4 and 1% of 1-(2-chloroethyl)-imidazolidin-2-one.

1-24. (canceled)

25. A compound of formula (I)
A₁-Q₁-Sₓ-Q₂-A₂ (I)
wherein:
A₁ and A₂ represent, independently of one another, an associative group comprising at least one nitrogen atom,
Q₁ and Q₂ represent, independently of one another, a bonding group,
x is an integer ranging from 3 to 6.

26. The compound as claimed in claim 25, wherein A₁ and A₂ are identical.

27. The compound as claimed in claim 25, wherein A₁ and A₂ are independently selected from the groups consisting of imidazolidinone, triazolyl, ureyl, bisureyl and ureidopyrimidyl.

28. The compound as claimed in claim 25, wherein A₁ and A₂ independently correspond to one of the following formulae (II) to (VI): 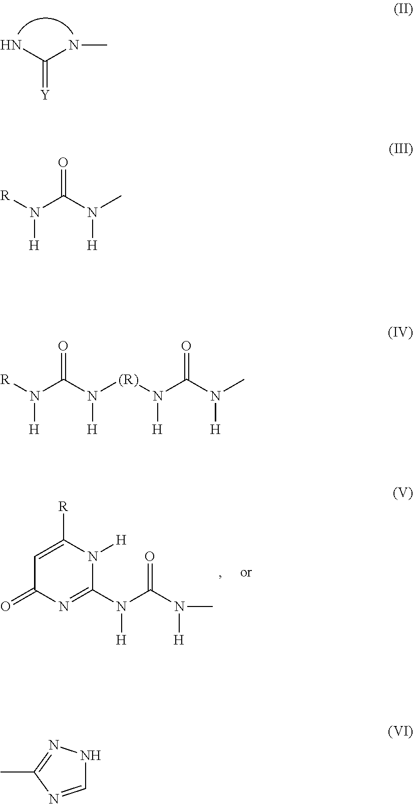
wherein:
R denotes a hydrocarbon-based group, and
Y denotes an oxygen or sulfur atom.

29. The compound as claimed in claim 25, wherein at least one of A₁ and A₂, is a group of formula (VII):

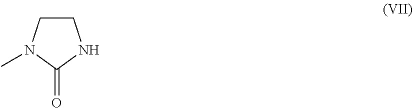

30. The compound as claimed in claim 25, wherein Q₁ and Q₂ are independently a linear or branched, substituted or unsubstituted, divalent C1-C24, hydrocarbon-based radical, optionally interrupted and/or substituted with one or more nitrogen or oxygen atoms.

31. The compound as claimed in claim 25, wherein x is equal to 4.

32. The compound as claimed in claim 25, selected from the group consisting of the compounds of following formulae (VIII) to (XI): 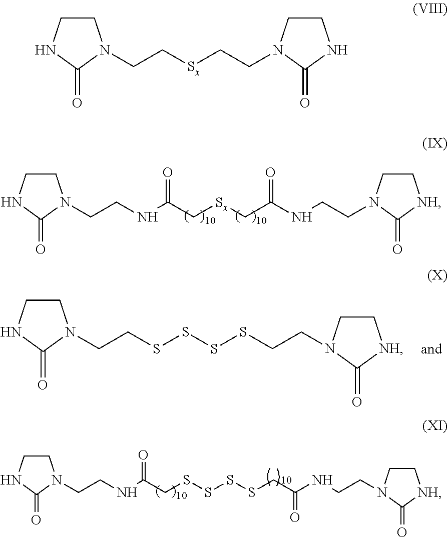
wherein x is an integer ranging from 3 to 4 in formulae (VIII) and (IX).

33. A mixture of different compounds of formula (I)
A₁-Q₁-Sₓ-Q₂-A₂ (I),
wherein:
A₁ and A₂ represent, independently of one another, an associative group comprising at least one nitrogen atom,
Q₁ and Q₂ represent, independently of one another, a bonding group,
x is an integer ranging from 2 to 6;
the compounds having different values of x and otherwise being identical,
wherein x has a mean value of between 2 and 6.

34. A process for preparing a compound as claimed claim 25, comprising a step of reacting a sulfur-containing compound with a compound of formula (XII)
A₁-Q₁-Z (XII)
and a compound of formula (XIII)
A₂-Q₂-Z (XIII),
wherein
A₁, A₂, Q₁ and Q₂ have the meanings defined in claim 25, and
Z represents a Cl atom or an SH group.

35. The process as claimed in claim 34, wherein the compound of formula (XII) and the compound of formula (XIII) are identical.

36. The process as claimed in claim 34, wherein the sulfur-containing compound is sodium tetrasulfide, Z is a Cl atom and the compound prepared is of formula (I) with x=4

37. The process as claimed in claim 34, wherein the sulfur-containing compound is sulfur monochloride, Z is an SH group and the compound prepared is of formula (I) with x=4.

38. The process as claimed in claim 34, wherein the sulfur-containing compound is sulfur, Z is an SH group and the compound prepared is of formula (I) with x ranging from 2 to 4.

39. A rubber composition comprising at least one diene elastomer, a reinforcing filler, a chemical crosslinking agent and a modifying agent, optionally already grafted onto the elastomer, said modifying agent being a compound as claimed in claim 25.

40. The composition as claimed in claim 39, wherein the diene elastomer comprises an essentially unsaturated diene elastomer comprising natural rubber, synthetic polyisoprenes, polybutadienes, butadiene copolymers, isoprene copolymers or mixtures thereof; and/or comprises an essentially saturated elastomer comprising butyl rubbers, diene/alpha-olefin copolymers, or mixtures thereof.

41. The composition as claimed in claim 39, wherein the chemical crosslinking agent comprises from 0.5 to 12 phr of sulfur, or from 0.01 to 10 phr of one or more peroxide compounds.

42. The composition as claimed in claim 39, wherein the content of modifying agent ranges from 0.01 to 50 mol %.

43. A process for preparing a rubber composition as claimed in claim 39, comprising one or more steps of thermomechanical kneading of the diene elastomer, the reinforcing filler, the chemical crosslinking agent and the modifying agent, and a step of extruding and calendering.

44. An item produced entirely or partly with a rubber composition as claimed in claim 39, the item comprising leaktight seals, thermal or acoustic insulators, cables, sheaths, footwear soles, packagings, coatings (paints, films, cosmetic products), patches (cosmetic or demopharmaceutical), other systems for trapping and releasing active agents, dressings, elastic clamp collars, vacuum pipes, or pipes and flexible tubing for the transportation of fluids.

45. A modified polymer obtained by grafting of a compound as claimed in claim 25.

46. The modified polymer as claimed in claim 45, the polymer being a diene elastomer.

47. The modified polymer as claimed in claim 45, the polymer being an essentially unsaturated diene elastomer selected from the group consisting of natural rubber, synthetic polyisoprenes, polybutadienes, butadiene copolymers, isoprene copolymers and mixtures of these elastomers; or an essentially saturated elastomer selected from the group consisting of butyl rubbers and diene/alpha-olefin copolymers.

48. A process for preparing a modified polymer, comprising a step of grafting a compound as claimed in claim 25 onto a polymer comprising at least one unsaturation.
